(19) United States
(12) Patent Application Publication
(10) Pub. No.: US 2008/0241151 A1
(43) Pub. Date: Oct. 2, 2008

(54) Virulence genes, proteins, and their use

(76) Inventor: Christoph Tang, London (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/893,649

(22) Filed: Aug. 17, 2007

Related U.S. Application Data
(63) Continuation of application No. 10/275,026, filed on Aug. 1, 2003.

(30) Foreign Application Priority Data
May 8, 2000 (GB) 0011108.8

(51) Int. Cl. A61K 39/395; C07K 14/00; C12N 15/11; C12N 1/20; A61K 39/00; G01N 33/00; C12Q 1/68; A61P 37/00; G01N 33/569; G01N 33/566; C07K 16/18; A61K 38/00; A61K 31/70; A61K 35/00

(52) U.S. Cl. 424/139.1; 530/350; 536/23.7; 435/252.3; 424/190.1; 424/250.1; 530/387.9; 514/12; 514/44; 424/93.2; 436/86; 436/501; 435/6; 435/7.32

(57) ABSTRACT

A series of genes from Neisseria meningitidis are shown to encode products which are implicated in virulence. The identification of these genes therefore allows attenuated microorganisms to be produced. Furthermore, the genes or their encoded products can be used in the manufacture of vaccines for therapeutic application.

### FIELD OF THE INVENTION

This invention relates to virulence genes and proteins, and their use. More particularly, it relates to genes and proteins/peptides obtained from Neisseria meningitidis, and their use in therapy and in screening for drugs.

### BACKGROUND OF THE INVENTION

Neisseria meningitidis is a Gram-negative bacterial pathogen that is implicated in septic shock and bacterial meningitis. This bacterium is a leading cause of bacterial meningitis in developed countries, and causes large-scale epidemics in Africa and China. In the UK, Neisseria meningitidis is the leading cause of death in childhood apart from road traffic accidents. The bacterium naturally inhabits the human naso-pharynx and then gains access to the blood stream from where it causes severe septicaemia or meningitis. Although current anti-microbials are effective in eliminating the bacterium from the body, the mortalilty from menigococcal septicaemia remains substantial. It would be desirable to provide means for treating or preventing conditions caused by Neisseria meningitidis, e.g. by immunisation.

### SUMMARY OF THE INVENTION

The present invention is based on the discovery of virulence genes in Neisseria meningitidis.

According to a first aspect of the invention, a peptide of the invention is encoded by an operon including any of the nucleotide sequences identified in claim 1, or a homologue thereof in a Gram-negative bacterium, or a functional fragment thereof, for therapeutic or diagnostic use.

The peptides may have many therapeutic uses for treating Neisseria infections, including use in vaccines for prophylactic application.

According to a second aspect, a polynucleotide encoding a peptide defined above, may also be useful for therapy or diagnosis.

According to a third aspect, the genes that encode the peptides may be utilised to prepare attenuated microorganisms. The attenuated microorganisms will usually have a mutation that disrupts the expression of one or more of the genes identified herein, to provide a strain that lacks virulence. These microorganisms will also have use in therapy and diagnosis.

According to a fourth aspect, the peptides, genes and attenuated microorganisms according to the invention may be used in the treatment or prevention of a condition associated with infection by Neisseria or Gram-negative bacteria.

### DESCRIPTION OF THE INVENTION

The present invention is based on the discovery of genes encoding peptides which are implicated in virulence. The peptides and genes of the invention are therefore useful for the preparation of therapeutic agents to treat infection. It should be understood that references to therapy also include preventative treatments, e.g. vaccination. Furthermore, while the products of the invention are intended primarily for treatment of infections in human patients, veterinary applications are also considered to be within the scope of the invention.

The present invention is described with reference to Neisseria meningitidis. However, all the Neisseria strains, and many other Gram-negative bacterial strains are likely to include related peptides or proteins having amino acid sequence identity or similarity to those identified herein. Organisms likely to contain the peptides include, but are not limited to the genera Salmonella, Enterobacter, Klebsiella, Shigella and Yersinia.

The experiments carried out to identify the virulence genes of the invention utilised N. meningitidis strain B. Homology searches were performed on the strain A database, however the proteins and genes from strain B are preferred.

Preferably, the peptides that may be useful in the various aspects of the invention have greater than a 40% similarity with the peptides identified herein. More preferably, the peptides have greater than 60% sequence similarity. Most preferably, the peptides have greater than 80% sequence similarity, e.g. 95% similarity. With regard to the polynucleotide sequences identified herein, related polynucleotides that may be useful in the various aspects of the invention may have greater than 40% identity with the sequences identified herein. More preferably, the polynucleotide sequences have greater than 60% sequence identity. Most preferably, the polynucleotide sequences have greater than 80% sequence identity, e.g. 95% identity.

The terms “similarity” and “identity” are known in the art. The use of the term “identity” refers to a sequence comparison based on identical matches between correspondingly identical positions in the sequences being compared. The term “similarity” refers to a comparison between amino acid sequences, and takes into account not only identical amino acids in corresponding positions, but also functionally similar amino acids in corresponding positions. Thus similarity between polypeptide sequences indicates functional similarity, in addition to sequence similarity.

Levels of identity between gene sequences and levels of identity or similarity between amino acid sequences can be calculated using known methods. In relation to the present invention, publicly available computer based methods for determining identity and similarity include the BLASTP, BLASTN and FASTA (Atschul et al., J. Molec. Biol., 1990; 215:403-410), the BLASTX program available from NCBI, and the Gap program from Genetics Computer Group, Madison Wis. The levels of similarity and identity provided herein, were obtained using the Gap program, with a Gap penalty of 12 and a Gap length penalty of 4 for determining the amino acid sequence comparisons, and a Gap penalty of 50 and a Gap length penalty of 3 for the polynucleotide sequence comparisons.

Having characterised a gene according to the invention, it is possible to use the gene sequence to search for related genes or peptides in other microorganisms. This may be carried out by searching in existing databases, e.g. EMBL or GenBank.

Peptides or proteins according to the invention may be purified and isolated by methods known in the art. In particular, having identified the gene sequence, it will be possible to use recombinant techniques to express the genes in a suitable host. Active fragments and related molecules can be identified and may be useful in therapy. For example, the peptides or their active fragments may be used as anfigenic determinants in a vaccine, to elicit an immune response. They may also be used in the preparation of antibodies, for passive immunisation, or diagnostic applications. Suitable antibodies include monoclonal antibodies, or fragments thereof, including single chain Fv fragments. Methods for the preparation of antibodies will be apparent to those skilled in the art.

Active fragments of the peptides are those that retain the biological function of the peptide. For example, when used to elicit an immune response, the fragment will be of sufficient size, such that antibodies generated from the fragment will discriminate between that peptide and other peptides on the bacterial microorganism. Typically, the fragment will be at least 30 nucleotides (10 amino acids) in size, preferably 60 nucleotides (20 amino acids) and most preferably greater than 90 nucleotides (30 amino acids) in size.

It should also be understood, that in addition to related molecules from other microorganisms, the invention encompasses modifications made to the peptides and polynucleotides identified herein which do not significantly alter the biological function. It will be apparent to the skilled person that the degeneracy of the genetic code can result in polynucleotides with minor base changes from those specified herein, but which nevertheless encode the same peptides. Complementary polynucleotides are also within the invention. Conservative replacements at the amino acid level are also envisaged, i.e. different acidic or basic amino acids may be substituted without substantial loss of function.

The preparation of vaccines based on attenuated microorganisms is known to those skilled in the art. Vaccine compositions can be formulated with suitable carriers or adjuvants, e.g. alum, as necessary or desired, to provide effective immunisation against infection. The preparation of vaccine formulations will be apparent to the skilled person. The attenuated microorganisms may be prepared with a mutation that disrupts the expression of any of the genes identified herein. The skilled person will be aware of methods for disrupting expression of particular genes. Techniques that may be used include insertional inactivation or gene deletion techniques. Attenuated microorganisms according to the invention may also comprise additional mutations in other genes, for example in a second gene identified herein or in a separate gene required for growth of the microorganism, e.g. an aro mutation or, with regard to Salmonella, in a gene located in the SPI2 region identified in WO-A-96/17951.

Attenuated microorganisms may also be used as carrier systems for the delivery of heterologous antigens, therapeutic proteins or nucleic acids (DNA or RNA). In this embodiment, the attenuated microorganisms are used to deliver a heterologous antigen, protein or nucleic acid to a particular site in vivo. Introduction of a heterologous antigen, peptide or nucleic acid into an attenuated microorganism can be carried out by conventional techniques, including the use of recombinant constructs, e.g. vectors, which comprise polynucleotides that express the heterologous antigen or therapeutic protein, and also include suitable promoter sequences. Alternatively, the gene that encodes the heterologous antigen or protein may be incorporated into the genome of the organism and the endogenous promoters used to control expression.

More generally, and as is well known to those skilled in the art, a suitable amount of an active component of the invention can be selected, for therapeutic use, as can suitable carriers or excipients, and routes of administration. These factors would be chosen or determined according to known criteria such as the nature/severity of the condition to be treated, the type and/or health of the subject etc.

In a separate embodiment, the products of the invention may be used in screening assays for the identification of potential antimicrobial drugs or for the detection for virulence. Routine screening assays are known to those skilled in the art, and can be adapted using the products of the invention in the appropriate way. For example, the products of the invention may be used as the target for a potential drug, with the ability of the drug to inactivate or bind to the target indicating its potential antimicrobial activity.

The various products of the invention may also be used in veterinary applications.

The following is a brief overview of the experimental procedure used to identify the virulence genes.

Signature-tagged mutagenesis (STM) (Hensel et al., Science, 1995; 269: 400-403) was used to identify genes in N. meningitidis that are essential for septicemic infection. In STM, individual mutants are tagged with unique sequence identifiers, allowing large numbers of mutants to be analyzed simultaneously. However, it is necessary to construct libraries of insertional mutants, so far a limitation in studying N. meningitidis. Mutagenesis was accomplished successfully using a method in which Neisseria DNA is modified in vitro using purified components of Tn 10 transposition. As N. meningitidis efficiently takes up exogenous DNA, the modified alleles are then introduced into N. meningitidis by transformation. The mutants can then be screened for their ability to cause systemic infection.

The vector pSTM115 (Sun et al. Nature Medicine, 2000; 6(11):1269-1273) was used as the transposon donor for in vitro mutagenesis. 96 pSTM115 derivatives, each containing unique signature tags, were included in 96 separate transposition reactions. The modified genomic DNA was repaired, and returned to the host by transformation. To determine whether Tn 10 insertion occurs at diverse sites, 40 transformants were assessed from a single transposition reaction by Southern blot analysis. Each had a single, distinct Tn 10 insertion. To establish whetherTn 10 integration was stable during systemic infection of infant rats, the hydridization patterns of six mutants before and after passage through rats were compared. Identical hybridization patterns before and after infection were obtained.

The experimental conditions used were as follows:

Bacterial strains and growth:

C311+ is an ET-5, serogroup B N. meningitidis isolate from a patient with invasive meningococcal infection (Virji et al., Mol. Microbiol., 1991; 5: 1831-1841). N. meningitidiswas grown on brain-heart infusion medium with 5% Levinthal's supplement. E. coli strains were propagated on Luria Bertani media. Kanamycin was added to solid media as required at concentrations of 75 and 50 μg/mi for N. meningitidis and E. coli, respectively.

In vitro transposition and insertion site characterization:

The pACYC184 origin of replication in pSTM115 was used to isolate the insertion site by marker rescue. Nucleotide sequencing was carried out using the dye-termination method (Perkin Elmer, Norwalk, Conn.) with primers NG62 (5′-TTGGTTAATTGGTTGTAACACTGG-3′) (SEQ ID NO. 209) or NG99 (5′-ATTCTCATGTTTGACAGCG-3′) (SEQ ID NO. 210). Homology searches were performed against protein databases (http://www.ncbi.nim.nih.gov/), including the serogroup A and B N.meningitidis and the N. gonorrhoeae genome sequences (http://www.sanger.ac.uk/Projects/N_meningitidis and http://www.tigr.org, and http://dnal.chem.ou.edu/gono.html, respectively). Tag amplification, cloning and Southern blot analyses:

Hybridizations and preparations of dot blots were performed as described in Hensel et aL, supra, except that tags were amplified with primers NG13 (5′-ATCCTACAACCTCAAGCT-3′) (SEQ ID NO. 211) and NG14 (5′-ATCCCATTCTAACCAAGC-3′) (SEQ ID NO. 212), and PCR products, rather than plasmid DNA, were fixed onto membranes. Oligonucleotides S1 (5′-AAGAGATTACGCGCAGACC-3′) (SEQ ID NO. 213) and S2 (5′-AATACGCAACCGCCTCTC-3′) (SEQ ID NO. 214) anneal to sequences in pSTM115 flanking the ‘signature tags’ and were used to amplify a 367-base-pair product from each pSTM115 derivative. For Southern blot analysis, the kanamycin-resistance cassettefrom pSTM115 was labelled using the random primers method (NEB), and was used as a probe against genomic DNA digested with Clal.

### Animal Model:

For screening the STM pools, mutants were grown individually for 18 h in microtiter plates. The bacteria were pooled, then re-suspended in PBS. Wistar rats (5 days old) were inoculated intraperitoneally with 100 μl of the suspension, and were monitored for 48 h. To establish the competitive index of a mutant, wild-type and mutant bacteria were grown for 18 h on solid media and collected into PBS, and rats were inoculated with a 1:1 ratio of mutant to wild-type cells in a total inoculum of 5×10⁶ CFU. The proportion of mutant (kanamycin-resistant) to wild-type (kanamycin-sensitive) bacteria was determined by plating replicate samples to media with or without added antibiotic.

The results of the homology searches are shown in Table 1.

TABLE 1

| SEQ ID NO. | PROTEIN | ACCESSION NO. | ORGANISM |
|---|---|---|---|
| 3 & 4 | 3′dehydroquinate synthase | NMB0647 | N. gonorrhoeae |
| 25 & 26 | Glycosyl transferase | LSI2 | N. gonorrheae |
| 35 & 36 | Polyribonucleotide nucleotidyl | NMB0758 | N. meningitidis |
| | transferase | | |
| 37 & 38 | Phosphoribosyl formyl glycinamide | PURL | E. coli |
| | synthase | | |
| 43 & 44 | Shikimate dehydrogenase | AROE | N. meningitidis |
| 47 & 48 | Hypothetical 21.7 kD protein | NMB0673 | N. meningitidis |
| 53 & 54 | Putative cell binding factor | NMB0345 | N. meningitidis |
| 57 & 58 | Hypothetical protein | HI0633 | H. influenzae |
| 61 & 62 | Na+/H+ antiporter | NMBN0536 | N. memingitidis |
| 63 & 64 | Chorismate synthase | AROC | V. anguillarum |
| 67 & 68 | Paraquat-inducible protein B | PQ15B | E. coli |
| 71 & 72 | 5′-methyltetrahydropteroylyl | NMB0944 | N. meningitidis |
| | triglutamate-homocysteine methyl | | |
| | transferase | | |
| 79 & 80 | α-1,2 N-acetylglucosamine | RFAK | N. meningitidis |
| | transferase | | |
| 83 & 84 | Putative RNA methylase | NMB1348 | N. meningitidis |
| 89 & 90 | L-lactate permease | NMB0543 | N. meningitidis |
| 91 & 92 | ABC transporter | NMB1240 | N. meningitidis |
| 103 & 104 | Probable GTP-binding protein | HI0393 | H. influenzae |
| 109 & 110 | Capsule polysaccharide modification | LIPB | N. meningitidis |
| | protein | | |
| 113 & 114 | Hypothetical 17.8 kD protein | NMB0734 | N. meningitidis |
| 115 & 116 | E. coli hypothetical protein | YIGC | S. typhimurium |
| 119 & 120 | Ribonuclease III | NMB0686 | N. meningitidis |
| 121 & 122 | AMPD protein | NMB0668 | N. meningitidis |
| 123 & 124 | 5-methyltetrahydropteroylyl | NMB0944 | N. meningitidis |
| | triglutamate-homocysteine methyl | | |
| | transferase | | |
| 133 & 134 | Putative ATP-depenent RNA | NMB1422 | N. meningitidis |
| | helicase | | |
| 137 & 138 | Putative RNA methylase | NMB1348 | N. meningitidis |
| 141 & 142 | Shikimate dehydrogenase | AROE | N. meningitidis |
| 143 & 144 | Putative outer membrane protein | OMPU | N. meningitidis |
| 145 & 146 | TONB protein | TONB | N. meningitidis |
| 147 & 148 | Putative apolipoprotein N-acyl | NMB0713 | N. meningitidis |
| | transferase | | |
| 149 & 150 | Transposase | NMB0991 | N. meningitidis |
| 153 & 154 | UTP-glucose-1-phosphate | NMB0638 | N. meningitidis |
| | uridylyltransferase | | |
| 157 & 158 | ADP Heptose-LPS heptosyl | NMB1527 | N. meningitidis |
| | transferase II | | |
| 161 & 162 | Putative membrane-bound lytic | NMB1279 | N. meningitidis |
| | murein transglycosylase B | | |
| 171 & 172 | Putative cell-binding factor | NMB0345 | N. meningitidis |
| 173 & 174 | P-amino benzoate synthetase | PABB | H. pylori J99 |
| 177 & 178 | 5′-methyltetrahydropteroylyl | NMB0944 | N. meningitidis |
| | triglutamate-homocysteine methyl | | |
| | transferase | | |
| 183 & 184 | Conserved hypothetical protein | NMB0183 | N. meningitidis |
| 185 & 186 | E. coli hypothetical protein | YIGC | S. typhi LT2 |

For the remaining sequences identified herein, no homology results were obtained.

The gene products were used to produce polyclonal antibodies that were tested in an Elisa assay against various N. meningitidis strains to evaluate their effectiveness as a vaccine candidate. The strains used were:
Neisseria meningitidis (B) Type 1000
Neisseria meningitidis (B) Type NGE31
Neisseria meningitidis (B) Type NGH15
Neisseria meningitidis (B) Type SW2 107
Neisseria meningitidis (B) Type NHG38
Neisseria meningitides (B) Type NGE28
Neisseria meningitides (B) Type 2996

This provides information as to the variety of N. meningitidis strains that are recognised by these antibodies.

N. Meningitidis was grown on Columbia agar with chocolated horse blood (Oxoid) for 14 hours at 37° C. in 5% CO₂. The cells were scraped from agar plates and resuspended in 20 ml PBS in a 50 ml tube. The cell suspension was heated for 30 minutes at 56° C. to kill the bacteria.

A 50 μl sample of the heat-killed N. Meningitidis was spread on the Columbia agar with the chocolated horse blood and incubated for 18 hours at 37° C., 5% CO₂. This allows confirmation that all N. Meningitidis cells have been killed. The OD₆₂₀ of the suspension Is adjusted to 0.1 OD units versus PBS.

Elisa with Heat Killed N. meningitidis

Elisa assays were carried out using the heat-killed N. meningitidis using the following protocol. Elisa plates were coated overnight with heat-killed cells (50 μl of killed bacteria in PBS to each well of 96 well plate and incubated 4° C.).

Standard Elisa protocols were followed, with all incubations at 37° C. for 1 hour. PBS/3% BSA blocking solution, PBS/Tween 0.1% wash solution, anti-rabbit AP conjugate secondary antibody (Sigma) and Sigma Fast P Nitrophenyl phosphate detection reagent (Sigma) were utilised. The data was read at 405 nm using an appropriate micro-titre plate reader. The sera used was that available seven days after the first booster vaccination (day 35 after first vaccination).

The antibodies tested were those raised against the gene products identified as SEQ ID NOS. 8, 102, 140, 158 and 202. In each case, the results showed that the anti-sera recognised several different strains of N. meningitidis B.

Ex vivo/in vitro Screening.

Protection against meningococcal disease in humans has been associated with the presence of bacteriocidal antibodies against N. meningitidis (Goldscheider et al. J. Exp. Med., 1969; 129: 1307-1326). There is also evidence to suggest a correlation between the presence of detectable bacteriocidal activity and protection in an in vivo model (reference Martin, J. Bacteriol., 2000; 83: 27-31). Therefore, the antiserum generated was used to evaluate the bacteriocidal activity of the antibodies generated.

The bactericidal assays were performed with pre-immune sera and the corresponding rabbit antiserum raised against the candidate antigens. Commercially available rabbit serum was used as the complement source following pre-screening to eliminate complement only killing. Dulbecco's PBS (Gibco) was used as a bufferwhere necessary. The N. meningitidis strain MC58, was grown at 37° C. (5% CO₂) for 14 hours prior to use in the assay.

200-400 CFU of MC58 in a 50 μl volume were incubated in the presence of complement (50 μl) with 100 μl of serial dilutions of heat-inactivated serum. Samples at time zero were plated to Columbia agar with chocolated horse blood (Oxoid). After incubation for 60 minutes, the number of surviving bacteria was evaluated by plating to Columbia agar with chocolated horse blood (Oxoid). The bacteriocidal activity was expressed in terms of percentage of bacteria surviving after 60 minutes. All samples were tested in duplicate and plated in triplicate. All appropriate positive and negative controls were utilised. In each test sample, the bacteriocidal activity was substantially greater than that for the pre-immune sera.

In vivo Screening.

To evaluate the protective efficacy of vaccine candidates, adult mice were immunised with the recombinant proteins identified herein as SEQ ID NOS. 102 and 108 and the protective response determined by live bacterial challenge. For each vaccine candidate 15 six week old mice (6 week old balb/C mice) were vaccinated (subcutaneously) with 25 μg of antigen on two separate occasions at three week intervals.

One week after the end of the immunisation schedule, the group was challenged with the homologous bacterial strain MC58. The bacteria were inoculated intraperiponeally in a volume of 500 μl in Brain Heart Infusion/0.5% iron dextran media at a dose of 10⁷ cfu. Previous results have shown that iron is required for initiation of bacteraemic disease in these animals. This model has previously been used to demonstrate the protective efficacy of vaccination (Lissolo et al., Infect. Immujn., 1995; 63: 884-890).

Control groups included animals vaccinated with adjuvant alone (negative control) or with adjuvant combined with purified PorA (positive control). PorA is an outer membrane protein expressed exclusively by N. meningitidis and is the principal target for bactericidal antibodies induced by outer membrane vesicle vaccines. Monoclonal antibodies against PorA have also been shown to passively protect animals in the infant rat model. PorA however varies considerably between strains and so while it elicits some protection when challenged with a homologous strain, it is not an ideal vaccine candidate. Survival was monitored following challenge. The negative control showed no survival after 48 hours. Those vaccinated with PorA showed 6 survivors at 72 hours. Those vaccinated with the proteins of SEQ ID NOS. 102 and 108 showed 5 and 3 survivors, respectively.

I claim:

1. An isolated peptide encoded by an operon comprising a nucleotide sequence selected from the group consisting of SEQ ID NOS. 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, of N. meningitidis, a related sequence obtainable from Gram-negative bacteria that has at least 60% sequence similarity or identity to any of the foregoing, or a functional fragment of any of the foregoing.

2. The peptide according to claim 1, wherein the sequence similarity or identity is at least 90%.

3. An isolated peptide comprising an amino acid sequence selected from the group consisting of SEQ ID NOS. 2, 4, 6, 8, 10, 12, 14, 16, 18, 20, 22, 24, 26, 28, 30, 32, 34, 36, 38, 40, 42, 44, 46, 48, 50, 52, 54, 56, 58, 60,62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, and 208, or a related sequence having at least 60% sequence similarity or identity to any of the foregoing, or a functional fragment of any of the foregoing.

4. An isolated polynucleotide encoding a peptide, wherein said polynucleotide comprises a nucleotide sequence selected from the group consisting of SEQ ID NOS. 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, of N. meningitidis, related sequences obtainable from Gram-negative bacteria that have at least 60% sequence similarity or identity, and functional fragments thereof.

5. A host microorganism transformed to express a peptide comprising an amino acid sequence selected from the group consisting of SEQ ID NOs. 2, 4, 6, 8, 10, 12, 14, 16, 18, 20, 22, 24, 26, 28, 30, 32, 34, 36, 38, 40, 42, 44, 46, 48, 50, 52, 54, 56, 58, 60,62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, and 208, or a related sequence having at least 60% sequence similarity or identity to any of the foregoing, or a functional fragment of any of the foregoing.

6. An attenuated microorganism comprising a mutation that disrupts the expression of a nucleotide sequence encoding a peptide, wherein said peptide comprises an amino acid sequence selected from the group consisting of SEQ ID NOs. 2, 4, 6, 8, 10, 12, 14, 16, 18, 20, 22, 24, 26, 28, 30, 32, 34, 36, 38, 40, 42, 44, 46, 48, 50, 52, 54, 56, 58, 60,62, 64, 66, 68, 70, 72, 74, 76, 78, 80, 82, 84, 86, 88, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 160, 162, 164, 166, 168, 170, 172, 174, 176, 178, 180, 182, 184, 186, 188, 190, 192, 194, 196, 198, 200, 202, 204, 206, and 208, or a related sequence having at least 60% sequence similarity or identity to any of the foregoing, or a functional fragment of any of the foregoing.

7. The attenuated microorganism of claim 6, wherein the nucleotide sequence is selected from the group consisting of SEQ ID NOS. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 159, 161, 163, 165, 167, 169, 171, 173, 175, 177, 179, 181, 183, 185, 187, 189, 191, 193, 195, 197, 199, 201, 203, 205, 207, of N. meningitidis, a related sequence obtainable from Gram-negative bacteria that has at least 60% sequence similarity or identity to any of the foregoing, or a functional fragment of any of the foregoing.

8. The microorganism according to claim 6, wherein the mutation is insertional inactivation or a gene deletion.

9. The microorganism according to claim 6, wherein the microorganism is Neisseria meningitidis.

10. The microorganism according to claim 6, comprising a second mutation in a second nucleotide sequence.

11. The microorganism according to claim 6, comprising a heterologous antigen, therapeutic peptide or nucleic acid.

12. A vaccine comprising an isolated peptide encoded by an operon comprising a nucleotide sequence selected from the group consisting of SEQ ID NOS. 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, of N. meningitidis, related sequence obtainable from Gram-negative bacteria and having at least 60% sequence similarity or identity, and functional fragments thereof; or the means for expressing said peptide.

13. A vaccine comprising an attenuated microorganism comprising a mutation that disrupts the expression of a polynucleotide encoding a peptide, wherein the peptide comprises an amino acid sequence selected from the group consisting of SEQ ID NOs. 2, 4, 6, 8, 10, 12, 14, 16, 18, 20, 22, 24, 26, 28, 30, 32, 34, 36, 38, 40, 42, 44, 46, 48, 50, 52, 54, 56, 58, 60,62, 64, 66, 68, 70, 72, 74, 76, 78, 80, 82, 84, 86, 88, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 160, 162, 164, 166, 168, 170, 172, 174, 176, 178, 180, 182, 184, 186, 188, 190, 192, 194, 196, 198, 200, 202, 204, 206, and 208, or a related sequence having at least 60% sequence similarity or identity to any of the foregoing, or a functional fragment of any of the foregoing.

14. The vaccine of claim 13, wherein said polynucleotide comprises a nucleotide sequence selected from the group consisting of SEQ ID NOS. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 159, 161, 163, 165, 167, 169, 171, 173, 175, 177, 179, 181, 183, 185, 187, 189, 191, 193, 195, 197, 199, 201, 203, 205, 207, of N. meningitidis, related sequences obtainable from Gram-negative bacteria that have at least 60% sequence similarity or identity, and functional fragments thereof.

15. An antibody raised against a peptide encoded by an operon comprising a nucleotide sequence selected from the group consisting of SEQ ID NOS. 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, of N. meningitidis, related sequence obtainable from Gram-negative bacteria and having at least 60% sequence similarity or identity at the peptide or nucleotide level, and functional fragments thereof.

16. A method for the treatment or prevention of a condition associated with infection by Neisseria or Gram-negative bacteria wherein said method comprises administering to a patient in need of such treatment or prevention a compound selected from one of the following:
(i) a peptide encoded by an operon comprising a nucleotide sequence selected from the group consisting of SEQ ID NOS. 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, of N. meningitidis, related sequences obtainable from Gram-negative bacteria that have at least 60% sequence similarity or identity, and functional fragments thereof;
(ii) a peptide comprising an amino acid sequence selected from the group consisting of SEQ ID NOS. 4, 6, 8, 10, 12, 14, 16, 18, 20, 22, 24, 26, 28, 30, 32, 34, 36, 38, 40, 42, 44, 46, 48, 50, 52, 54, 56, 58, 60,62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206 and 208;
(iii) a polynucleotide encoding a peptide wherein said polynucleotide comprises a nucleotide sequence selected from the group consisting of SEQ ID NOS. 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, of N. meningitidis, related sequences obtainable from Gram-negative bacteria that have at least 60% sequence similarity or identity, and functional fragments thereof;
(iv) a host transformed to express a peptide encoded by an operon comprising a nucleotide sequence selected from the group consisting of SEQ ID NOS. 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, of N. meningitidis, related sequences obtainable from Gram-negative bacteria that have at least 60% sequence similarity or identity, and functional fragments thereof;
(v) an attenuated microorganism comprising a mutation that disrupts the expression of a nucleotide sequence encoding a peptide comprising an amino acid sequence selected from the group consisting of SEQ ID NOs. 2, 4, 6, 8, 10, 12, 14, 16, 18, 20, 22, 24, 26, 28, 30, 32, 34, 36, 38, 40, 42, 44, 46, 48, 50, 52, 54, 56, 58, 60,62, 64, 66, 68, 70, 72, 74, 76, 78, 80, 82, 84, 86, 88, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 160, 162, 164, 166, 168, 170, 172, 174, 176, 178, 180, 182, 184, 186, 188, 190, 192, 194, 196, 198, 200, 202, 204, 206, and 208, or a related sequence having at least 60% sequence similarity or identity to any of the foregoing, or a functional fragment of any of the foregoing.
(vi) an attenuated microorganism comprising a mutation that disrupts the expression of a nucleotide sequence selected from the group consisting of SEQ ID NOS. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 159, 161, 163, 165, 167, 169, 171, 173, 175, 177, 179, 181, 183, 185, 187, 189, 191, 193, 195, 197, 199, 201, 203, 205, 207, of N. meningitidis, related sequences obtainable from Gram-negative bacteria that have at least 60% sequence similarity or identity, and functional fragments thereof;
(vii) an antibody raised against a peptide encoded by an operon comprising a nucleotide sequence selected from the group consisting of SEQ ID NOS. 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, of N. meningitidis, related sequence obtainable from Gram-negative bacteria and having at least 60% sequence similarity or identity at the peptide or nucleotide level, and functional fragments thereof; and
(viii) a polynucleotide encoding a peptide wherein said peptide comprises an amino acid sequence selected from the group consisting of SEQ ID NOS. 4, 6, 8, 10, 12, 14, 16, 18, 20, 22, 24, 26, 28, 30, 32, 34, 36, 38, 40, 42, 44, 46, 48, 50, 52, 54, 56, 58, 60,62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206 and 208.

17. The method according to claim 16, wherein the condition is meningitis.

18. The method according to claim 16, for treating an animal.

19. A screening assay for the identification of an antimicrobial drug wherein said assay uses one or more of the following:
(i) a peptide encoded by an operon comprising a nucleotide sequence selected from the group consisting of SEQ ID NOS. 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, of N. meningitidis, related sequences obtainable from Gram-negative bacteria that have at least 60% sequence similarity or identity, and functional fragments thereof;
(ii) a peptide comprising an amino acid sequence selected from the group consisting of SEQ ID NOS. 4, 6, 8, 10, 12, 14, 16, 18, 20, 22, 24, 26, 28, 30, 32, 34, 36, 38, 40, 42, 44, 46, 48, 50, 52, 54, 56, 58, 60,62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206 and 208;
(iii) a polynucleotide encoding a peptide wherein said polynucleotide comprises a nucleotide sequence selected from the group consisting of SEQ ID NOS. 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, of N. meningitidis, related sequences obtainable from Gram-negative bacteria that have at least 60% sequence similarity or identity, and functional fragments thereof;
(iv) a host transformed to express a peptide encoded by an operon comprising a nucleotide sequence selected from the group consisting of SEQ ID NOS. 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, of N. meningitidis, related sequences obtainable from Gram-negative bacteria that have at least 60% sequence similarity or identity, and functional fragments thereof;
(v) an attenuated microorganism comprising a mutation that disrupts the expression of a nucleotide sequence encoding a peptide comprising an amino acid sequence selected from the group consisting of SEQ ID NOs. 2, 4, 6, 8, 10, 12, 14, 16, 18, 20, 22, 24, 26, 28, 30, 32, 34, 36, 38, 40, 42, 44, 46, 48, 50, 52, 54, 56, 58, 60,62, 64, 66, 68, 70, 72, 74, 76, 78, 80, 82, 84, 86, 88, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 160, 162, 164, 166, 168, 170, 172, 174, 176, 178, 180, 182, 184, 186, 188, 190, 192, 194, 196, 198, 200, 202, 204, 206, and 208, or a related sequence having at least 60% sequence similarity or identity to any of the foregoing, or a functional fragment of any of the foregoing.
(vi) an attenuated microorganism comprising a mutation that disrupts the expression of a nucleotide sequence selected from the group consisting of SEQ ID NOS. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 159, 161, 163, 165, 167, 169, 171, 173, 175, 177, 179, 181, 183, 185, 187, 189, 191, 193, 195, 197, 199, 201, 203, 205, 207, of N. meningitidis, related sequences obtainable from Gram-negative bacteria that have at least 60% sequence similarity or identity, and functional fragments thereof;
(vii) an antibody raised against a peptide encoded by an operon comprising a nucleotide sequence selected from the group consisting of SEQ ID NOS. 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, of N. meningitidis, related sequence obtainable from Gram-negative bacteria and having at least 60% sequence similarity or identity at the peptide or nucleotide level, and functional fragments thereof; and
(viii) a polynucleotide encoding a peptide wherein said peptide comprises an amino acid sequence selected from the group consisting of SEQ ID NOS. 4, 6, 8, 10, 12, 14, 16, 18, 20, 22, 24, 26, 28, 30, 32, 34, 36, 38, 40, 42, 44, 46, 48, 50, 52, 54, 56, 58, 60,62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206 and 208.

20. An isolated polynucleotide encoding a peptide, wherein said peptide comprises an amino acid sequence selected from the group consisting of SEQ ID NOS. 2, 4, 6, 8, 10, 12, 14, 16, 18, 20, 22, 24, 26, 28, 30, 32, 34, 36, 38, 40, 42, 44, 46, 48, 50, 52, 54, 56, 58, 60,62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170 172, 174, 176, 178, 180, 182, 184, 186, 188, 190, 192, 194, 196, 198, 200, 202, 204, 206 and 208.
